(19) United States
(12) Patent Application Publication
(10) Pub. No.: US 2001/0016673 A1
(43) Pub. Date: Aug. 23, 2001

(54) Method of producing olefins and feedstocks for use in olefin production from crude oil having low pentane insolubles and high hydrogen content

(75) Inventors: Robert S. Bridges, Friendswood, TX (US); Richard B. Halsey, Houston, TX (US); Donald H. Powers, McKinney, TX (US)

Correspondence Address: Kevin M. Carroll, LYONDELL CHEMICAL COMPANY, 3801 West Chester Pike, Newtown Square, PA, 19073, US

(73) Assignee: EQUISTAR CHEMICALS, L.P.

(21) Appl. No.: 09/810,801

(22) Filed: Mar. 16, 2001

Related U.S. Application Data
(63) Continuation of application No. 09/290,011, filed on Apr. 12, 1999.

(51) Int. Cl. C07C 4/04

(52) U.S. Cl. 585/648; 585/649; 585/650; 585/652; 208/130; 208/131

(57) ABSTRACT

Olefins may be produced by thermally steam cracking a crude oil having pentane insolubles less than or equal to 1.2, ASTM D-893, and a weight percent of hydrogen greater than or equal to 12.5.

### FIELD OF THE INVENTION

The invention relates to a method of producing olefins from whole crude oil. In a preferred embodiment, the feedstock contains short residuum having a boiling point greater than 650° C.

### BACKGROUND OF THE INVENTION

The production of low molecular weight hydrocarbon products for use as chemicals or chemical intermediates at high temperatures is well documented. For instance, U.S. Pat. No. 2,846,360 discloses a conversion process wherein a stream of high temperature catalytically inert particulate solids is contacted with petroleum hydrocarbons. In this process, vaporous conversion products are passed from a chemical coking zone countercurrent to the stream of coal particulate solids, the higher boiling portion being condensed on the coal solids.

A more simplified process pertains to steam cracking wherein liquid hydrocarbon feedstocks are cracked in the presence of steam to produce lower molecular weight olefins. Typically, such processes require the feedstock to be deasphalted and hydrotreated prior to feeding the feedstock into the steam cracking unit. For example, see U.S Pat. No. 4,257,871.

Less costly means for producing olefins is desired.

### SUMMARY OF THE INVENTION

It is an object of this invention to provide a process for producing olefins from a whole crude oil feedstock. The crude oil is characterized by a hydrogen content in excess of 12.5 weight percent and pentane insolubles, ASTM D-893, less than or equal to 1.2. The feedstock may be subjected to a thermal or steam cracking operation.

In a preferred embodiment, the crude oil feedstock may further be characterized as containing short residuum.

### BRIEF DESCRIPTION OF THE DRAWINGS

FIG. 1 is a schematic flow diagram of a pilot plant thermal steam cracking apparatus used in the process of the invention.

FIG. 2 is a boiling, point distillation curve of the Alaskan crude oil of Example 1.

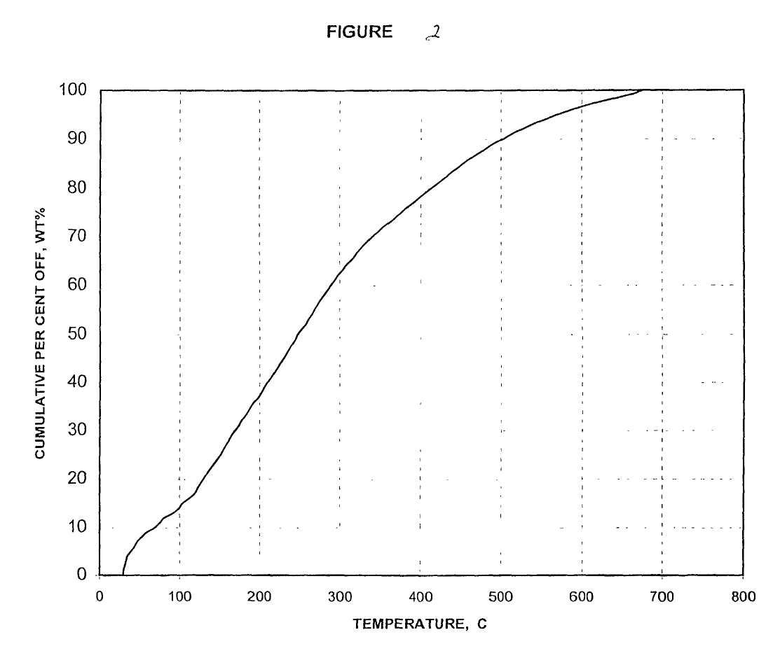

### DETAILED DESCRIPTION OF THE INVENTION

Olefins are produced in accordance with the invention from crude oil feedstocks. The term “crude oil feedstock” as used herein denotes a full range of crude oils having pentane insolubles, ASTM D-893, less than or equal to 1.2, and a hydrogen content in excess of 12.5 weight percent. The term may include primary, secondary or tertiary recoveries of conventional or offshore oil fields as well as the myriad of feedstocks derived therefrom as well as “syncrudes” such as those that can be derived from coal, shale oil, tar sands and bitumens. The crude oil feedstock may be virgin (straight run) or generated synthetically by blending.

Olefins may be produced in accordance with the invention by thermal cracking with steam the crude oil feedstock. The temperature within the reactor during this thermal cracking operation is between from about 500° C. to about 1100° C., preferably between about 700° C. up to about 900° C. The pressure employed may range from about 0 psig up to about 100 psig.

The crude oil feedstock may further contain short residuum. Short residuum, sometimes referred to as vacuum residuum, is defined as that portion of the crude oil feedstock which has a boiling point of from about 565° C. to the final boiling point of the crude oil feedstock. In one embodiment of the invention, up to 100 weight percent of the short residuum has a boiling point greater than or equal to 650° C. Generally, when present, the amount of short residuum in the crude oil feedstock is greater than 2 percent.

The crude oil feedstock of the invention further has pentane insolubles, ASTM D-893, less than or equal to 1.2, preferably less than or equal to 1.0, most preferably less than 0.5. In addition, the weight percent of hydrogen of the crude oil feedstock is greater than or equal to 12.5, preferably greater than or equal to 12.7, most preferably greater than 13.0.

Since the crude oil feedstock has pentane insolubles of less than 1.2, ASTM D-893, it need not be subjected to deasphalting prior to being fed into the steam cracking furnace.

In addition, since the crude oil feedstock of the invention has hydrogen content in excess of 12.5 weight percent, it need not be subjected to hydrotreatment prior to being fed into the steam cracking furnace.

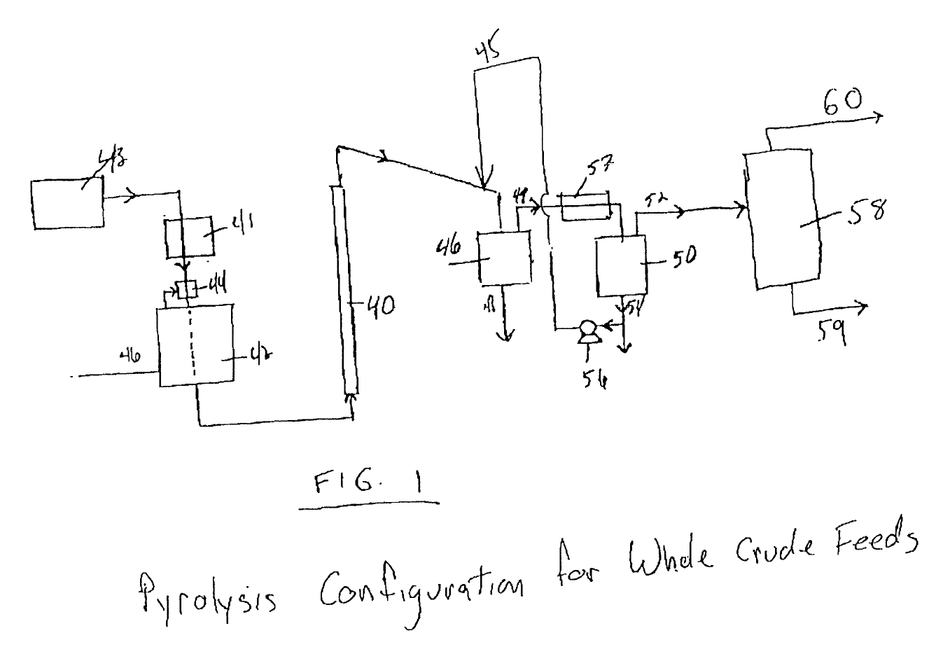

Referring now to FIG. 1, a crude oil feedstock defined by the invention may be converted into desirable olefin products. Thermal cracking tube 40 may be made of Incoloy (800HT). A section of the pipe is heated by electric furnace to about 700° to about 850° C. The furnace may have a multitude of independently controlled heating zones.

Before entering thermal cracking tube 40, the olefin feedstock enters heater 41 from olefin feedstock entry port 43. The feedstock is heated in heater 41 to a temperature of from 200° to about 260° C. The hydrocarbon is then injected at the top center of mixing chamber 44 and mixed with steam, which enters the chamber from side 46, both radially and tangentially, to promote thorough mixing, at a temperature of about 480° to about 600° C. About 0.3 to about 2.0 kg steam per kg of hydrocarbon is used. Proper mixing of the steam and hydrocarbon is often critical to the successful operation of the cracking tube.

The steam/oil mixture is further preferably heated (electrically) to a temperature sufficient to fully vaporize the hydrocarbon in heater 42 before entering thermal cracking tube 40. The flow rates of the oil and steam streams may be chosen to give a 0.05 to about 0.5 second residence time of the vaporized components in cracking tube 40, at a cracking tube temperature of about 700 to about 850° C. In cracking tube 40, the feedstock is converted to the desirable light olefin products, as well as by-product liquids.

After exiting cracking tube 40, the steam/hydrocarbon mixture is diluted with quench water and oil (at 45) in order to rapidly lower the temperature (down to about 200° to about 300° C.) of the effluent stream to reduce secondary condensation reactions. The diluted product is directed into a separator vessel 46 where the majority of the fuel oil and water is withdrawn as liquid phase 48. The remaining vapor stream 49 is further cooled and additional liquids, including water, are separated in vessels 50 and 58 after passing through heat exchanger 57. The lighter compounds that do not condense are removed as vapor stream at 60 while the liquid components are collected at 54 and 59. Pump 56 recirculates liquid effluent 54 back to 45.

### EXAMPLE 1

A sample of Alaskan crude oil was obtained from ARCO. This material is labeled as “Feed Q”. It was not necessary to fractionate or treat this whole crude oil in any way. It was found to have the characteristics given in Table 1.

TABLE 1

| | |
|---|---|
| API Gravity, ASTM D4052 | 39.2 |
| Sulfur, ASTM D2622 | 0.27 wt % |
| Conradson Carbon, ASTM D4530 | 1.26 wt % |
| Hydrogen Content, ASTM D4880 | 13.2 wt % |
| Pentane Insolubles, ASTM D893 | 0.25 wt % |

Boiling Point Distribution

| | |
|---|---|
| Initial Boiling Point (IBP) to 200° C. | 37.4 wt % |
| 200-340° C. | 32.6 wt % |
| 340-540° C. | 22.1 wt % |
| 540-565° C. | 2.6 wt % |
| 565-590° C. | 1.4 wt % |
| 590-625° C. | 1.7 wt % |
| 625-650° C. | 0.9 wt % |
| 650+° C. | 1.3 wt % |

The boiling point distribution for Feed Q is presented graphically in FIG. 3. The whole crude oil was used as feed to a thermal steam cracking apparatus as depicted in FIG. 1 and as described previously. Feed Q was fed to the thermal cracking tube at a rate of 4.1 kg/hr. Feed Q was blended with 4.9 kg/hr steam and the mixture was further heated to 538° C. The mixture was fed to the thermal cracking tube (having 1.25 cm diameter and 762 cm in length in the heated zone) that was maintained at 829° C. external tube surface temperature. The mass fraction and the chemical analysis of the vapor stream was used to determine the distribution of products as set forth in Table 2.

TABLE 2

| | |
|---|---|
| Hydrogen | 0.6 wt % |
| Methane | 8.9 wt % |
| Ethylene | 19.3 wt % |
| Acetylene | 0.2 wt % |
| Ethane | 2.6 wt % |
| Propylene | 12.2 wt % |
| Propane | 0.7 wt % |
| Methylacetylene/Propadiene | 0.5 wt % |
| 1,3 Butadiene | 4.7 wt % |
| Other C₄ Compounds | 5.7 wt % |
| Higher Molecular Weight Compounds | 44.6 wt % |

### EXAMPLE2

Feed Q, whose physical properties are listed in Table 1, was used as feed to a thermal steam cracker at a different set of conditions. Feed Q was fed to the thermal cracking tube at a rate of 4.1 kg/hr. Feed Q was blended with 4.9 kg/hr steam and the mixture was further heated to 538° C. The mixture was fed to the thermal cracking tube (of Example 1) that was maintained at 843° C. external tube surface temperature. The mass fraction and the chemical analysis of the vapor stream was used to determine the distribution of products as set forth in Table 3.

TABLE 3

| | |
|---|---|
| Hydrogen | 0.7 wt % |
| Methane | 9.3 wt % |
| Ethylene | 20.4 wt % |
| Acetylene | 0.3 wt % |
| Ethane | 2.4 wt % |
| Propylene | 12.1 wt % |
| Propane | 0.6 wt % |
| Methylacetylene/Propadiene | 0.5 wt % |
| 1,3 Butadiene | 4.7 wt % |
| Other C₄ Compounds | 5.1 wt % |
| Higher Molecular Weight Compounds | 43.9 wt % |

From the foregoing description, one skilled in the art can easily ascertain the essential characteristics of this invention, and without departing from the spirit and scope thereof, can make various changes and modifications of the invention to adapt it to various usages and conditions.

1. A process for producing olefins which comprises thermally steam cracking a crude oil wherein:
(A) the crude oil has pentane insolubles, ASTM D893, less than or equal to 1.2; and
(B) the weight percent hydrogen of the crude oil is greater than 12.5.

2. The process of claim 1, wherein the weight percent hydrogen of the crude oil is greater than or equal to 12.7.

3. The process of claim 2, wherein the weight percent hydrogen of the crude oil is between about 12.7 and about 13.8.

4. The process of claim 1, wherein the crude oil further contains short residuum.

5. The process of claim 4, wherein at least 3 percent of the short residuum has a boiling point greater than or equal to 650° C.

6. The process of claim 1, wherein the crude oil has pentane insolubles, ASTM D-893, less than or equal to 1.0.

7. The process of claim 3, wherein the crude oil has pentane insolubles, ASTM D-893, less than or equal to 1.0.

8. The process of claim 4, wherein between about 20 to about 60 weight percent of the short residuum has a boiling point in excess of 650° C.

9. The process of claim 4, wherein the crude oil has pentane insolubles less than or equal to 1.0.

10. The process of claim 4, wherein the crude oil contains from about 2 to about 50 weight percent of a short residuum.

11. The process of claim 1, wherein the crude oil feedstock has pentane insolubles, ASTM D893, less than or equal to 0.30.

12. A process for producing olefins which comprises subjecting a crude oil feedstock to steam and heat for a time sufficient to render olefins, wherein the crude oil feedstock contains a short residuum and wherein the pentane insolubles, ASTM D 893, of the crude oil feedstock is less than or equal to 1.2 and the hydrogen content of the crude oil feedstock is at least 12.5 weight percent.

13. The process of claim 12, wherein at least 3 percent of the short residuum has a boiling point in excess of 650° C.

14. The process of claim 13, wherein about at least 6 weight percent of the short residuum has a boiling point in excess of 650° C.

15. The process of claim 12, wherein about 10 to about 45 percent of the short residuum has a boiling point in excess of 650° C.

16. The process of claim 12, wherein the cede oil feedstock has at least 12.7 weight percent of hydrogen.

17. The process of claim 16, wherein the crude oil feedstock has between about 12.7 to about 13.8 weight percent of hydrogen.

18. The process of claim 12, wherein the crude oil feedstock has pentane insolubles less than 1.0.

19. The process of claim 12, wherein between from about 2 to about 50 weight percent of the crude oil feedstock is short residuum.

20. The process of claim 12, wherein the crude oil feedstock has pentane insolubles, ASTM D893, less than or equal to 0.30.
